(19) United States
(12) Patent Application Publication
(10) Pub. No.: US 2023/0135505 A1
(43) Pub. Date: May 4, 2023

(54) BIO-BASED POLYURETHANE DISPERSIONS FOR DECORATIVE COSMETIC APPLICATIONS

(71) Applicant: Covestro Deutschland AG, Leverkusen (DE)

(72) Inventors: Sophie Viala, Köln (DE); Laurence Pottie, Köln (DE); Sebastian Doerr, Düsseldorf (DE); Igor Pochorovski, Bergisch Gladbach (DE)

(21) Appl. No.: 17/912,548

(22) PCT Filed: Mar. 26, 2021
(86) PCT No.: PCT/EP2021/057872

§ 371 Date: Sep. 19, 2022

(30) Foreign Application Priority Data
Mar. 31, 2020 (EP) 20167029.6

(51) Int. Cl. A61K 8/87; A61Q 1/10; A61Q 1/02; A61K 8/04; C08G 18/12; C08G 18/75; C08G 18/42; C08G 18/32; C08G 18/66

(52) U.S. Cl. CPC A61K 8/87; A61Q 1/10; A61Q 1/02; A61K 8/04; C08G 18/12; C08G 18/755; C08G 18/758; C08G 18/4241; C08G 18/3234; C08G 18/6651; A61K 2800/43

(57) ABSTRACT

The invention relates to decorative cosmetic compositions containing specific polyurethanes or aqueous dispersions thereof and containing constituents that produce decorative effects.

The present invention relates to decorative cosmetic compositions containing specific polyurethanes or aqueous dispersions thereof and constituents that provide decorative effects.

A decorative cosmetic composition such as that of the invention serves for the decorative, especially colored, styling of human skin, mucous membranes, semi-mucous membranes, nails, especially fingernails or toenails, and hair, especially eyelids and eyebrows. The decorative effect is achieved by at least one effect-imparting component. The decorative composition according to the invention can, for example, be a face make-up (foundation), a tinted (day) cream, a blusher, a rouge, a mascara, an eyeliner, kohl, an eye shadow, a lipstick, a lip gloss. These special cosmetic formulations are used to change the color or to apply make-up to the body, for example to cover up dark rings under the eyes, an uneven complexion or other skin imperfections such as redness, spots, wrinkles or pimples, thus giving the user a more esthetic appearance. The aforementioned list of decorative products is of course non-limiting.

The decorative cosmetic compositions generally comprise one or more dyes selected, for example, from the group consisting of soluble dyes, inorganic pigments such as iron oxides, chromium oxides, ultramarine, manganese violet, organic pigments and nacre. Depending on the formulation form, such decorative cosmetic compositions may consist of up to 80% by weight of colorants and fillers, based on the total weight of the composition.

Consumers naturally desire a long-lasting decorative effect when using decorative cosmetic formulations. In particular, consumers expect good resistance to water, such as during bathing or showering, and to tears or sweat, especially during sports activities.

The stability of decorative products to water, tears or sweat (often called water resistance) is improved using film-forming polymers. Preferred film-forming polymers selected are polymers based on acrylates or vinylpyrrolidones. The disadvantages of such film-forming polymers are known to the person skilled in the art. One is that the acrylate polymers form hard and brittle films. This results in an unpleasant feeling while wearing the product. Because of the sticky skin feel, the vinylpyrrolidones can only be used in limited concentrations.

The use of polyurethane dispersions is also known in decorative cosmetics. For instance, US 2007/0154440 describes the use of a film-forming polyurethane having a molecular weight of at least 50 000 in a cosmetic formulation for the creation of a long-lasting film on the skin. FR 2832058 describes the use of an aqueous polyurethane dispersion in an eyeliner composition. US 20070025943 describes the combination of a film-forming (meth)acrylate copolymer and a film-forming polyurethane in a cosmetic composition. EP 0775483 (DE 69621104) describes the use of an aqueous dispersion of synthetic, film-forming polymer particles in a composition for making up the lips. EP 1010418 describes the use of an aqueous polyurethane dispersion in a wax-free mascara composition. WO 2003039445 describes the use of an aqueous polyurethane dispersion in a cosmetic composition. WO02070577A1 (US 2004/0197293) describes anionic polyurethanes that can be used in cosmetic compositions. However, it does not describe any concrete examples of cosmetic compositions. The anionic polyurethanes described have a comparatively low water resistance and form aqueous polyurethane dispersions of comparatively high viscosity, making their processing more difficult.

There is therefore still room for improvement in terms of wear comfort, in particular reduced stickiness, stability, in particular water resistance and gloss of the polyurethane-containing cosmetic compositions, especially for providing decorative effects, from the prior art. Furthermore, the aqueous polyurethane dispersions used in the prior art often have a disadvantageously high viscosity, which can make it difficult to process them or to incorporate them into cosmetic formulations.

The present invention accordingly has the object of providing a decorative cosmetic composition which contains components, in particular polyurethanes, that originate to a large extent from renewable sources. Furthermore, the decorative cosmetic composition is to have a high wear comfort, in particular reduced stickiness, a high stability, in particular abrasion resistance, and improved gloss properties. Furthermore, the aqueous polyurethane dispersions used in accordance with the invention are to have a comparatively low viscosity so that they can be easily incorporated into cosmetic compositions for decorative purposes.

Surprisingly, the object is achieved by the use of specific polyurethanes or aqueous dispersions thereof, obtainable by reacting one or more water-insoluble, non-water-dispersible, isocyanate-functional polyurethane prepolymers A with one or more amino-functional compounds B), characterized in that the polyurethane prepolymer A) is obtainable by reacting one or more polyester polyols having a glass transition temperature T_{g} of at least −50° C. and one or more polyisocyanates.

The present invention thus provides a decorative cosmetic composition, containing at least one polyurethane obtainable by reacting one or more water-insoluble, non-water-dispersible, isocyanate-functional polyurethane prepolymers A with one or more amino-functional compounds B), characterized in that the polyurethane prepolymer A) is obtainable by reacting one or more polyester polyols having a glass transition temperature T_{g} of at least −50° C. and one or more polyisocyanates.

Preferably, the present invention provides a decorative cosmetic composition according to the invention, containing at least one polyurethane obtainable by reacting one or more isocyanate-functional polyurethane prepolymers A) that essentially have neither ionic nor ionogenic groups, with one or more amino-functional compounds B).

In the context of the invention, the term “water-insoluble, non-water-dispersible polyurethane prepolymer” means in particular that the water solubility of the prepolymer used in accordance with the invention at 23° C. is less than 10 g/liter, more preferably less than 5 g/liter, and the prepolymer at 23° does not result in a sedimentation-stable dispersion in water, especially deionized water. In other words, the prepolymer settles out when an attempt is made to disperse it in water.

Preferably, the polyurethane prepolymer A) used in accordance with the invention has terminal isocyanate groups, meaning that the isocyanate groups are at the chain ends of the prepolymer. All chain ends of a polymer particularly preferably have isocyanate groups.

Furthermore, the polyurethane prepolymer A) used in accordance with the invention preferably essentially has neither ionic nor ionogenic (i.e. capable of forming ionic groups) groups, i.e. the content of ionic and ionogenic groups is expediently below 15 milliequivalents per 100 g of polyurethane prepolymer A), preferably below 5 milliequivalents, particularly preferably below one milliequivalent and especially preferably below 0.1 milliequivalent per 100 g of polyurethane prepolymer A).

The amino-functional compounds B) are preferably selected from primary and/or secondary amines and/or diamines More particularly, the amino-functional compounds B) comprise at least one diamine. The amino-functional compounds B) are preferably selected from amino-functional compounds B2) that have ionic or ionogenic groups, and amino-functional compounds B1) that do not have any ionic or ionogenic groups.

In a particularly preferred embodiment of the invention, the amino-functional compounds B) comprise at least one amino-functional compound B2) that has ionic and/or ionogenic groups. Particular preference is given to the use, as ionic and/or ionogenic group, of the sulfonate and/or sulfonic acid group, more preferably the sodium sulfonate group.

In a further preferred embodiment of the invention, the amino-functional compounds B) comprise both amino-functional compounds B2) that have ionic and/or ionogenic groups, and amino-functional compounds B1) that do not have any ionic and/or ionogenic groups.

In the context of the invention, polyurethanes are accordingly polymeric compounds having at least two, preferably at least three, urethane group-containing repeating units:

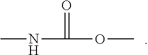

According to the invention, such polyurethanes are also included which, due to the production process, also comprise urea group-containing repeating units:

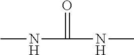

as are formed in particular during the reaction of the isocyanate-terminated prepolymers A) with the amino-functional compounds B).

The decorative cosmetic compositions according to the invention may also be water-containing, i.e. aqueous compositions in which the polyurethane is dispersed, i.e. present in essentially undissolved form. Alongside other liquid media that are optionally present, for example solvents, water may be the main constituent (>50% by weight) of the dispersion media, based on the total amount of the liquid dispersion media in the cosmetic compositions according to the invention, and possibly even the sole liquid dispersion medium.

The decorative cosmetic compositions according to the invention preferably have a content of volatile organic compounds (VOCs) of less than 80% by weight, more preferably of less than 55% by weight, more preferably still of less than 40% by weight, based on the decorative cosmetic composition.

The aqueous polyurethane dispersions used to produce the decorative cosmetic compositions according to the invention preferably have a content of volatile organic compounds (VOCs) of less than 10% by weight, more preferably of less than 3% by weight, more preferably still of less than 1% by weight, based on the aqueous polyurethane dispersion.

In the context of the present invention, the content of volatile organic compounds (VOCs) is especially determined by gas chromatography analysis.

The water-insoluble and non-water-dispersible, isocyanate-functional polyurethane prepolymers used in accordance with the invention essentially have neither ionic nor ionogenic groups. The water insolubility or lack of dispersibility in water relates to deionized water without addition of surfactants. In the context of the present invention, this means that the proportion of the ionic and/or ionogenic groups, such as anionic groups in particular, such as carboxylate or sulfate, or of cationic groups is less than 15 milliequivalents per 100 g of polyurethane prepolymer A), preferably less than 5 milliequivalents, particularly preferably less than one milliequivalent and very particularly preferably less than 0.1 milliequivalent per 100 g of polyurethane prepolymer A).

In the case of acidic ionic and/or ionogenic groups, the acid number of the prepolymer is appropriately below 30 mg KOH/g of prepolymer, preferably below 10 mg KOH/g of prepolymer. The acid number indicates the mass of potassium hydroxide in milligrams required to neutralize 1 g of the sample to be examined, measurement to DIN EN ISO 211. The neutralized acids, i.e. the corresponding salts, naturally have a zero or reduced acid number. What is crucial here in accordance with the invention is the acid number of the corresponding free acid.

The prepolymers A) used to produce the polyurethanes are obtainable by reacting one or more polyester polyols having a glass transition temperature T_{G} of at least −50° C. and one or more polyisocyanates. The one or more polyester polyols used to produce the prepolymers A) more preferably have a glass transition temperature T_{g} of −50 to 0° C., particularly preferably −40 to −10° C., determined in each case by DSC measurement in accordance with DIN 65467, with a heating rate of 20 K/min.

The polyurethanes used in accordance with the invention preferably have a glass transition temperature Tg of at least −50° C., particularly preferably −50 to 0° C., especially preferably −30 to 0° C., very particularly preferably −20 to −10° C., determined in each case by DSC measurement in accordance with DIN 65467, with a heating rate of 20 K/min.

Preferably, one or more polyurethanes are used according to the invention, at least 50% by weight of the components used to form the polyurethane(s), in particular the polyester polyol(s), more preferably the dicarboxylic acids and/or dihydroxy compounds used to form the polyester polyol(s), originating from renewable sources. The resulting polymer, which is based to an extent of at least 50% by weight on bio-based raw materials, can be qualified as “naturally derived” (“ingredients of natural origin”) according to the standard ISO 16128-1.

More preferably in accordance with the invention, the term “from renewable sources” means that a source is selected for the relevant material which to an extent of at least 90% by weight, preferably at least 95% by weight, especially preferably at least 99% by weight of the material that is identified as “from renewable sources” from plant or fermentation processes in which only living organisms and plants take part in the fermentation process.

The polyurethane(s) used in accordance with the invention are preferably made from renewable sources, i.e. bio-based, to an extent of at least 30 mol %, particularly preferably at least 40 mol %, especially preferably at least 50 mol %.

The present invention also relates to a polyurethane obtainable by reacting one or more water-insoluble, non-water-dispersible, isocyanate-functional polyurethane prepolymers A with one or more amino-functional compounds B), characterized in that the polyurethane prepolymer A) is obtainable by reacting one or more polyester polyols having a glass transition temperature T_{g} of at least −50° C. and one or more polyisocyanates. What has been said in relation to the composition according to the invention correspondingly applies to the polyurethanes according to the invention.

The polyurethanes present in the decorative cosmetic compositions according to the invention as a result of the prepolymer A) contain one or more of the above-mentioned polyester polyols of bio-based origin having the specific glass transition temperature described. In addition, the prepolymers present according to the invention may contain at least one sequence selected from the group consisting of polyether, polycarbonate, polyether-polycarbonate and further polyester sequences. According to the invention, this can mean that the polyurethanes contain ether group- and/or carbonate group-containing and ester group repeating units. The polyurethanes present according to the invention for example contain exclusively polyester sequences based on the above-described polyester polyols having a glass transition temperature T_{g} of at least −50° C. However, they may additionally include polyether and polycarbonate sequences as are formed for example when producing polycarbonate polyols using polyether diols. In addition, they may include polyether-polycarbonate sequences which result from the use of polyether-polycarbonate polyols, as described in more detail hereinafter.

Polyurethanes preferred according to the invention are obtained using polymeric polyester polyols having a glass transition temperature T_{g} of at least −50° C., which have number-average molecular weights of preferably about 400 to about 6000 g/mol, these and subsequent reported molecular weights being determined by gel permeation chromatography against polystyrene standard in tetrahydrofuran at 23° C. Their use in the production of the polyurethanes or polyurethane prepolymers leads as a result of reaction with polyisocyanates to the formation of corresponding polyester sequences in the polyurethanes with a corresponding molar weight of these sequences. Particular preference is given to polyurethanes according to the invention which are obtained from polymeric polyester polyols having a glass transition temperature T_{g} of at least −50° C. with a linear structure and optionally additionally polyether diols and/or polymeric polycarbonate diols and/or polyether-polycarbonate polyols or further polyester polyols.

The polyurethanes according to the invention are preferably substantially linear molecules, but may also be branched, although this is less preferred.

The number-average molecular weight of the polyurethanes preferably used according to the invention is for example approximately from 1000 to 200 000 g/mol, preferably from 5000 to 150 000 g/mol.

The polyurethanes present in the decorative cosmetic compositions according to the invention are added to the said compositions in particular as aqueous dispersions.

Preferred polyurethanes or polyurethane dispersions to be used in accordance with the invention are obtainable by producing

A) isocyanate-functional prepolymers from
A1) organic polyisocyanates,
A2) polymeric polyester polyols having a glass transition temperature T_{g} of at least −50° C., preferably having number-average molecular weights of 400 to 8000 g/mol, these and subsequent reported molecular weights being determined by gel permeation chromatography against polystyrene standard in tetrahydrofuran at 23° C., more preferably of 400 to 6000 g/mol and particularly preferably of 600 to 3000 g/mol, and OH functionalities of preferably 1.5 to 6, more preferably 1.8 to 3, particularly preferably of 1.9 to 2.1,
A3) optionally hydroxy-functional compounds having molecular weights of preferably 62 to 399 g/mol, and
A4) optionally non-ionic hydrophilizing agents, and
B) wholly or partly reacting the free NCO groups thereof
with one or more amino-functional compounds B), such as primary and/or secondary amines and/or diamines.

The polyurethanes used in accordance with the invention are preferably dispersed in water before, during or after step B).

Particular preference is given in step B) to the reaction with a diamine or two or more diamines with chain extension. In addition, monofunctional amines may be added as chain terminators to control the molecular weight.

As component B), it is in particular possible to use amines that do not have any ionic or ionogenic groups, such as anionically hydrophilizing groups, hereinafter component B1), and it is possible to use amines that have ionic or ionogenic groups, such as in particular anionically hydrophilizing groups, hereinafter component B2).

Preferably, in step B) of the reaction of the prepolymer, a mixture of component B1) and component B2) is reacted. The use of component B1) can result in formation of a high molar mass without a rise in the viscosity of the isocyanate-functional prepolymer produced beforehand to a degree that would be a barrier to processing. The use of the combination of components B1) and B2) can establish an optimal balance between hydrophilicity and chain length and hence a pleasant skin feel.

The polyurethanes used in accordance with the invention preferably have anionic groups, preferably sulfonate groups. These anionic groups are introduced into the polyurethanes used in accordance with the invention via the amine component B2) reacted in step B). The polyurethanes used in accordance with the invention optionally additionally include non-ionic components for hydrophilization. Particularly preferably, the polyurethanes used in accordance with the invention, for hydrophilization, contain exclusively sulfonate groups which are introduced into the polyurethane via corresponding diamines as component B2).

In order to achieve good sedimentation stability, the number-average particle size of the specific polyurethane dispersions is preferably less than 750 nm, particularly preferably less than 500 nm, determined by means of laser correlation spectroscopy after dilution with deionized water, instrument used: Malvern Zetasizer 1000, Malvern Inst. Limited).

The solids content of the polyurethane dispersions, which is preferably used to produce the decorative cosmetic composition of the invention, is generally 10% to 70% by weight, preferably 30% to 65% by weight, particularly preferably 30% to 50% by weight. The solids contents are ascertained according to the invention by heating a weighed sample to 125° C. to constant weight. At constant weight, the solids content is calculated by reweighing the sample.

Preferably, these polyurethane dispersions include less than 5% by weight, particularly preferably less than 0.2% by weight, based on the mass of the dispersions, of unbound organic amines. The content in the decorative cosmetic compositions is accordingly even lower.

Suitable polyisocyanates of component A1) are in particular the aliphatic, aromatic or cycloaliphatic polyisocyanates having an NCO functionality of greater than or equal to 2 that are known per se to those skilled in the art.

Examples of such suitable polyisocyanates include butylene 1,4-diisocyanate, hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any isomer content, cyclohexylene 1,4-diisocyanate, 4-isocyanatomethyloctane 1,8-diisocyanate (nonane triisocyanate), phenylene 1,4-diisocyanate, tolylene 2,4- and/or 2,6-diisocyanate, naphthylene 1,5-diisocyanate, diphenylmethane 2,2′- and/or 2,4′- and/or 4,4′-diisocyanate, 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) having C1-C8-alkyl groups.

In addition to the aforementioned polyisocyanates, it is also possible to use modified diisocyanates having a functionality ≥2, with uretdione, isocyanurate, urethane, allophanate, biuret, iminooxadiazinedione or oxadiazinetrione structure and also mixtures of proportions of these.

Preference is given in accordance with the invention to polyisocyanates or polyisocyanate mixtures of the aforementioned type having exclusively aliphatically or cycloaliphatically bonded isocyanate groups or mixtures thereof and an average NCO functionality of the mixture of 2 to 4, preferably of 2 to 2.6 and particularly preferably of 2 to 2.4, very particularly preferably 2.

Particularly preferably employed in A1) are hexamethylene diisocyanate, isophorone diisocyanate or the isomeric bis(4,4′-isocyanatocyclohexyl)methanes and also mixtures of the abovementioned diisocyanates.

Employed in A2) are polymeric polyester polyols having a glass transition temperature T_{g} of at least −50° C. and having a number-average molecular weight Mₙ of preferably 400 to 8000 g/mol, more preferably of 400 to 6000 g/mol and particularly preferably of 600 to 3000 g/mol. These preferably have an OH functionality of 1.5 to 6, more preferably of 1.8 to 3, most preferably of 1.9 to 2.1.

The expression “polymeric” polyester polyols here means in particular that the polyols mentioned have at least two, preferably at least three, repeating units bonded to one another.

These polyester polyols according to the invention having a glass transition temperature T_{g} of at least −50° C. may be used singly or in any desired mixtures with one another in A2). Further polyols which may optionally additionally be present are the following, which are known per se to those skilled in the art: polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols.

The polyester polyols used in accordance with the invention are the conventional polycondensates of di- and optionally tri- and tetraols and di- and optionally tri- and tetracarboxylic acids or hydroxycarboxylic acids or lactones. For the production of the polyesters it is also possible to use, instead of the free polycarboxylic acids, the corresponding polycarboxylic anhydrides or corresponding polycarboxylic esters of lower alcohols.

Examples of suitable diols are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate, preference being given to hexane-1,6-diol and isomers, butane-1,4-diol, neopentyl glycol and neopentyl glycol hydroxypivalate. In addition, it is also possible to use polyols such as trimethylolpropane, glycerol, erythritol, pentaerythritol, trimethylolbenzene or trishydroxyethyl isocyanurate.

The dicarboxylic acids used may be phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, succinic acid, suberic acid, 2-methylsuccinic acid, 3,3-diethylglutaric acid and/or 2,2-dimethylsuccinic acid. It is also possible to use the corresponding anhydrides as the acid source.

Polyols having more than two OH groups are described above. Instead of or in addition to this, it is also possible to use polyols such as trimethylolpropane, glycerol, erythritol, pentaerythritol, trimethylolbenzene or trishydroxyethyl isocyanurate. Provided that the average functionality of the polyol to be esterified is >2, it is additionally also possible to use monocarboxylic acids, such as benzoic acid and hexanecarboxylic acid, as well.

Preferred acids are aliphatic or aromatic, particularly preferably aliphatic, acids of the above-mentioned type. Particular preference is given to succinic acid, adipic acid, isophthalic acid and phthalic acid.

Succinic acid, which is preferably used for the production of the polyester polyols used in accordance with the invention, is preferably obtained from renewable sources. In this case, succinic acid is produced, for example, by fermentation of starch or biomass, as described for example in DE 10 2008 051727 A1 and DE 10 2007 019184. Preferably, according to the invention, at least 50% by weight of the succinic acid used originates from renewable sources. Furthermore or in addition, it is also possible for at least a portion of the dihydroxy compounds used in the polyester polyol according to the invention to originate from renewable sources and hence to increase the proportion of polyurethane components that originate from renewable sources. The polyester polyol used in accordance with the invention preferably contains at least one dihydroxy compound selected from butane-1,4-diol, propane-1,3-diol, isopropanediol, hexane-1,6-diol, ethylene glycol, which more preferably originate from renewable sources, and mixtures thereof.

Examples of hydroxycarboxylic acids that may be used as co-reactants in the production of a polyester polyol having terminal hydroxyl groups include hydroxycaproic acid, hydroxybutyric acid, hydroxydecanoic acid, hydroxystearic acid and the like. Suitable lactones are caprolactone, butyrolactone and homologs. Preference is given to caprolactone.

As component A2) for producing the polyurethanes, particular preference according to the invention is given to polyester polyols having a glass transition temperature T_{g} of at least −50° C. and having a number-average molecular weight of 600 to 3000 g/mol, especially aliphatic polyester polyols based on aliphatic carboxylic acids and aliphatic polyols, in particular based on succinic acid or adipic acid and aliphatic alcohols such as butane-1,4-diol, hexane-1,6-diol and/or neopentyl glycol.

Besides the polyester polyol described as component A2) above, further polyols may optionally be present in the polyurethane used in accordance with the invention, for example hydroxyl-containing polycarbonates, preferably polycarbonate diols, having number-average molecular weights Mₙ of preferably 400 to 8000 g/mol, preferably 600 to 3000 g/mol. These are obtainable by reacting carbonic acid derivatives, such as diphenyl carbonate, dimethyl carbonate or phosgene, with polyols, preferably diols.

Examples of such diols are ethylene glycol, propane-1,2- and -1,3-diol, butane-1,3- and -1,4-diol, hexane-1,6-diol, octane-1,8-diol, neopentyl glycol, 1,4-bishydroxymethylcyclohexane, 2-methylpropane-1,3-diol, 2,2,4-trimethylpentane-1,3-diol, dipropylene glycol, polypropylene glycols, dibutylene glycol, polybutylene glycols, bisphenol A and lactone-modified diols of the abovementioned type.

It is preferable when the diol component comprises 40% to 100% by weight of hexanediol, preference being given to hexane-1,6-diol and/or hexanediol derivatives. Such hexanediol derivatives are based on hexanediol and have not only terminal OH groups but also ester groups or ether groups. Such derivatives are obtainable by reaction of hexanediol with excess caprolactone or by etherification of hexanediol with itself to afford di- or trihexylene glycol.

Polyether-polycarbonate diols may also be used instead of or in addition to pure polycarbonate diols.

Hydroxyl-containing polycarbonates preferably have a linear structure.

It is likewise possible to use polyether polyols in addition to the polyester polyols according to the invention.

Particularly suitable examples are the polytetramethylene glycol polyethers known per se in polyurethane chemistry, as obtainable by cationic ring-opening polymerization of tetrahydrofuran.

Likewise suitable polyether polyols are the addition products, known per se, of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and/or polybutylene glycols, are employable, especially with the abovementioned preferred molecular weights.

Suitable starter molecules that may be used are all compounds known from the prior art, for example water, butyldiglycol, glycerol, diethylene glycol, trimethylolpropane, propylene glycol, sorbitol, ethylenediamine, triethanolamine, butane-1,4-diol.

Particularly preferred polyols that may be present in addition to component A2) are polytetramethylene glycol polyethers and polycarbonate polyols or mixtures thereof, polytetramethylene glycol polyethers being particularly preferred.

In a preferred embodiment of the present invention, it is possible to use, in addition to the polyester polyol according to the invention (component A2), the following polyol mixtures: Mixtures containing at least one polyether polyol and at least one polycarbonate polyol, mixtures containing more than one polyether polyol, or a mixture of two or more polyether polyols having different molecular weights, these in particular being poly(tetramethylene glycol) polyether polyols (such as (HO—(CH₂—CH₂—CH₂—CH₂—O)ₓ—H), mixtures containing more than one polyether polyol and at least one polycarbonate polyol and also the above-mentioned polyester polyols, the polyol component by definition essentially having neither ionic nor ionogenic groups.

As component A3) it is optionally possible to use polyols, especially nonpolymeric polyols, of said preferred molecular weight range from 62 to 399 mol/g having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol and any desired mixtures thereof with one another, especially neopentyl glycol.

Also suitable are ester diols of the stated molecular weight range, such as α-hydroxybutyl ε-hydroxycaproate, ω-hydroxyhexyl γ-hydroxybutyrate, β-hydroxyethyl adipate or bis(β-hydroxyethyl) terephthalate.

Monofunctional isocyanate-reactive compounds containing hydroxyl groups can also be used as component A3). Examples of such monofunctional compounds are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

In a preferred embodiment of the invention, the polyurethane used in accordance with the invention comprises less than about 10% by weight of component A3), preferably less than 5% by weight of component A3), based in each case on the total mass of the polyurethane, even more preferably component A3) is not used for the production of the polyurethane.

As component A4), one or more, in particular isocyanate-reactive, non-ionic hydrophilizing agents are optionally used for the production of the polyurethanes used in accordance with the invention. The hydrophilizing agents used as component A4) differ in particular from components A2) and A3).

Suitable non-ionically hydrophilizing compounds as component A4) are, for example, polyoxyalkylene ethers having isocyanate-reactive groups, such as hydroxyl, amino or thiol groups. Preference is given to monohydroxy-functional polyalkylene oxide polyether alcohols having a statistical average of 5 to 70, preferably 7 to 55, ethylene oxide units per molecule, as obtainable in a manner known per se by alkoxylation of suitable starter molecules (for example in Ullmanns Encyclopädie der technischen Chemie [Ullmann's Encyclopedia of Industrial Chemistry], 4th edition, volume 19, Verlag Chemie, Weinheim p. 31-38). These are either pure polyethylene oxide ethers or mixed polyalkylene oxide ethers, and they contain at least 30 mol %, preferably at least 40 mol %, of ethylene oxide units, based on all alkylene oxide units present.

Particularly preferred non-ionic compounds are monofunctional mixed polyalkylene oxide polyethers having 40 to 100 mol % of ethylene oxide units and 0 to 60 mol % of propylene oxide units.

Suitable starter molecules for such nonionic hydrophilizing agents are especially saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or olein alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and heterocyclic secondary amines such as morpholine, pyrrolidine, piperidine or 1H-pyrazole. Preferred starter molecules are saturated monoalcohols of the abovementioned type. It is particularly preferable to use diethylene glycol monobutyl ether or n-butanol as starter molecules.

Alkylene oxides suitable for the alkoxylation reaction are in particular ethylene oxide and propylene oxide, which can be used in the alkoxylation reaction in any desired sequence or else in a mixture.

Component B) is preferably selected from primary or secondary amines and/or diamines. It especially comprises diamines.

As component B), it is in particular possible to use amines that do not have any ionic or ionogenic groups, such as anionically hydrophilizing groups (hereinafter component B1)), and it is possible to use amines that have ionic or ionogenic groups, such as in particular anionically hydrophilizing groups (hereinafter component B2)). Preferably, in step B) of the reaction of the prepolymer, a mixture of component B1) and component B2) is reacted.

For example, components B1) used may be organic di- or polyamines such as for example ethylene-1,2-diamine, 1,2- and 1,3-diaminopropane, 1,4-diaminobutane, 1,6-diaminohexane, isophoronediamine, an isomeric mixture of 2,2,4- and 2,4,4-trimethylhexamethylenediamine, 2-methylpentamethylenediamine, diethylenetriamine, 4,4-diaminodicyclohexylmethane, hydrazine hydrate and/or dimethylethylenediamine.

In addition, components B1) used may also be compounds that have not only a primary amino group but also secondary amino groups, or not only an amino group (primary or secondary) but also OH groups. Examples thereof are primary/secondary amines such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Components B1) used may further be monofunctional isocyanate-reactive amine compounds, such as for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines formed from diprimary amines and monocarboxylic acids, monoketime of diprimary amines, primary/tertiary amines, such as N,N-dimethylaminopropylamine.

Components B1) used are preferably ethylene-1,2-diamine, bis(4-aminocyclohexyl)methane, 1,4-diaminobutane, isophoronediamine, ethanolamine, diethanolamine and diethylenetriamine.

Component B) particularly preferably comprises at least one component B2). Suitable anionically hydrophilizing compounds as component B2) preferably contain a sulfonic acid group or sulfonate group, particularly preferably a sodium sulfonate group. Suitable anionically hydrophilizing compounds as component B2) are especially the alkali metal salts of mono- and diaminosulfonic acids. Examples of such anionic hydrophilizing agents are salts of 2-(2-aminoethylamino)ethanesulfonic acid, ethylenediaminepropylsulfonic or ethylenediaminebutylsulfonic acid, propylene-1,2- or -1,3-diamine-β-ethylsulfonic acid or taurine. The salt of cyclohexylaminopropanesulfonic acid (CAPS) from WO-A 01/88006 can also be used as an anionic hydrophilizing agent.

Particularly preferred anionic hydrophilizing agents B2) are those that comprise sulfonate groups as ionic groups and two amino groups, such as the salts of 2-(2-aminoethylamino)ethylsulfonic acid and propylene-1,3-diamine-β-ethylsulfonic acid.

The polyurethanes used in accordance with the invention particularly preferably comprise at least one sulfonate group.

The anionic group in component B2) may optionally also be a carboxylate or carboxylic acid group.

In that case, component B2) is preferably selected from diaminocarboxylic acids. However, this embodiment is less preferred since carboxylic acid-based components B2) have to be used in higher concentrations.

Mixtures of anionic hydrophilizing agents B2) and non-ionic hydrophilizing agents A4) may also be used for hydrophilization.

In a preferred embodiment for production of the specific polyurethane dispersions the components A1) to A4) and B1) to B2) are used in the following amounts, where the individual amounts always add up to 100% by weight:

5% to 40% by weight of component A1),

55% to 90% by weight of A2),

0.5% to 20% by weight of the sum total of components A3) and/or B1)

0.1% to 25% by weight of the sum total of components A4) and/or B2), with particular preference being given to using 0.1% to 5% by weight of anionic or potentially anionic hydrophilizing agents B2), based on the total amounts of components A1) to A4) and B1) to B2).

In a particularly preferred embodiment for production of the specific polyurethane dispersions the components A1) to A4) and B1) to B2) are used in the following amounts, where the individual amounts always add up to 100% by weight:

5% to 35% by weight of component A1),

60% to 90% by weight of A2),

0.5% to 15% by weight of the sum total of components A3) and/or B1)

0.1% to 15% by weight of the sum total of components A4) and/or B2), with particular preference being given to using 0.2% to 4% by weight of anionic or potentially anionic hydrophilizing agents B2), based on the total amounts of components A1) to A4) and B1) to B2).

In a very particularly preferred embodiment for production of the specific polyurethane dispersions the components A1) to A4) and B1) to B2) are used in the following amounts, where the individual amounts always add up to 100% by weight:

10% to 30% by weight of component A1),

65% to 85% by weight of A2),

0.5% to 14% by weight of the sum total of components A3) and/or B1)

0.1% to 13.5% by weight of the sum total of components A4) and/or B2), with particular preference being given to using 0.5% to 3.0% by weight of anionic or potentially anionic hydrophilizing agents of B2), based on the total amounts of components A1) to A4) and B1) to B2).

According to the invention, very particular preference is given to using polyurethanes bearing the INCI names Polyurethane-93 and/or Polyurethane-99. The INCI name is an international nomenclature for cosmetic ingredients. INCI stands for “International Nomenclature of Cosmetic Ingredients”.

The production of the polyurethane dispersions may be carried out in one or more stages in homogeneous phase or, in the case of a multistage reaction, partially in disperse phase. Complete or partial performance of polyaddition from A1) to A4) is preferably followed by a dispersing, emulsifying or dissolving step. This is optionally followed by a further polyaddition or modification in disperse phase.

It is possible here to use any methods known from the prior art, for example prepolymer mixing methods, acetone methods or melt dispersion methods. Preference is given to employing the acetone method.

For production by the acetone method, it is customary to form an initial charge including all or some of constituents A2) to A4) and the polyisocyanate component A1) for production of an isocyanate-functional prepolymer, and optionally to dilute them with a solvent that is water-miscible but inert toward isocyanate groups, and heat them to temperatures in the range from 50° C. to 120° C. The isocyanate addition reaction can be accelerated using the catalysts known in polyurethane chemistry.

Suitable solvents are the customary aliphatic keto-functional solvents, such as acetone, 2-butanone, which can be added not just at the start of the production but optionally also in portions at a later stage. Acetone and 2-butanone are preferred and acetone is particularly preferred. The addition of other solvents without isocyanate-reactive groups is also possible, but not preferred.

Subsequently, any constituents of A1) to A4) not added at the start of the reaction are added.

In the production of the polyurethane prepolymer from A1) to A4), the molar ratio of isocyanate groups to isocyanate-reactive groups is generally 1.05 to 3.5, preferably 1.1 to 3.0, particularly preferably 1.1 to 2.5.

Components A1) to A4) are converted partly or fully to the prepolymer, but preferably fully. Polyurethane prepolymers containing free isocyanate groups are thus obtained in neat form or in solution.

In the neutralization step, partial or complete conversion of potentially anionic groups to anionic groups is accomplished using bases such as tertiary amines, e.g. trialkylamines having 1 to 12 and preferably 1 to 6 carbon atoms, particularly preferably 2 to 3 carbon atoms in each alkyl radical, or very particularly preferably alkali metal bases such as the corresponding hydroxides. The use of organic amines is not preferred.

Usable neutralizing agents preferably include inorganic bases such as aqueous ammonia solution or sodium or potassium hydroxide.

Preference is given to sodium hydroxide and potassium hydroxide.

The molar amount of the bases is between 50 and 125 mol %, preferably between 70 and 100 mol %, of the molar amount of the acid groups to be neutralized. The neutralization can also be effected simultaneously with the dispersing when the dispersion water already contains the neutralizing agent.

Subsequently, if not yet effected or only partially effected, the prepolymer obtained is dissolved using aliphatic ketones such as acetone or 2-butanone in a further process step.

Components A1) to A4) are converted partly or fully to the prepolymer, but preferably fully. Polyurethane prepolymers containing free isocyanate groups are thus obtained in neat form or in solution.

In the chain extension of stage B), NH₂- and/or NH-functional components are reacted with the still remaining isocyanate groups of the prepolymer. It is preferable when the chain extension/termination is carried out prior to the dispersing in water.

Suitable components B) for chain extension are particularly organic di- or polyamines B1) such as ethylenediamine, 1,2- and 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylenetriamine, diaminodicyclohexylmethane and/or dimethylethylendiamine.

Also employable are compounds B1) that have not only a primary amino group but also secondary amino groups, or not only an amino group (primary or secondary) but also OH groups. Examples of these are primary/secondary amines such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine for chain extension or termination are used.

Chain termination is typically accomplished using amines B1) having an isocyanate-reactive group, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines formed from diprimary amines and monocarboxylic acids, monoketimes of diprimary amines, primary/tertiary amines, such as N,N-dimethylaminopropylamine.

When anionic hydrophilizing agents according to definition B2) having NH₂ groups or NH groups are used for chain extension, the chain extension of the prepolymers is preferably effected prior to the dispersing.

The degree of chain extension, i.e. the equivalents ratio of NCO-reactive groups of the compounds used for chain extension and chain termination to free NCO groups of the prepolymer, is generally between 40% and 150%, preferably between 50% and 110%, particularly preferably between 60% and 100%.

The aminic components B1) and B2) may optionally be used in water- or solvent-diluted form in the process of the invention, individually or in mixtures, any sequence of addition being possible in principle.

When water or organic solvents are included as a diluent, the diluent content in the component for chain extension used in B) is preferably from 40% to 95% by weight.

The dispersing preferably follows the chain extension. To this end, the dissolved and chain-extended polyurethane polymer is either introduced into the dispersion water, optionally under high shear, for example vigorous stirring, or, conversely, the dispersion water is stirred into the chain-extended polyurethane polymer solutions. It is preferable when the water is added to the dissolved, chain-extended polyurethane polymer.

The solvent still present in the dispersions after the dispersion step is typically then removed by distillation. Removal even during the dispersing is likewise possible.

The residual content of organic solvents in the polyurethane dispersions thus produced is typically less than 10% by weight, preferably less than 3% by weight, based on the total dispersion.

The pH of the aqueous polyurethane dispersions used in accordance with the invention is typically less than 8.0, preferably less than 7.5, and is particularly preferably between 5.5 and 7.5.

The decorative cosmetic composition according to the invention preferably contains 0.1% to 20% by weight of the above-described polyurethane and especially 0.5% to 10% by weight, based in each case on the total weight of the composition.

The decorative cosmetic composition of the invention is used for decorative, in particular colored or effect-imparting styling of human skin, mucous membrane, semi-mucous membrane, hair, in particular the eyelids and the eyebrows, more preferably not the head hair, and nails, in particular toenails or fingernails. The decorative, i.e. color effect or other effect, for example glitter effect, metallic effect etc., is achieved by at least one effect-imparting, in particular color- and/or effect-imparting constituent. The decorative composition according to the invention can, for example, be a face make-up, for example foundation, a tinted cream, in particular day cream, a blusher, a rouge, a mascara, an eyeliner, kohl, an eye shadow, a lipstick, a lip gloss; nail polishes. A characteristic of the decorative cosmetic compositions is generally that they are so-called “leave on” products, which at least partially remain on the skin or hair after application.

The decorative cosmetic composition according to the invention can in particular be solid, liquid or semisolid. For example, the composition may be present in the form of aqueous pigment dispersion, oil-in-water, water-in-oil, water-in-silicone oil, silicone oil-in-water, oil-in-water-in-oil, water-in-oil-in-water or solid emulsions, i.e. emulsions stabilized by solids such as Pickering emulsions. The formulation according to the invention may also be foamed with a propellant gas. The formulation according to the invention can also be in the form of loose powder, compact powder, foam (so-called mousse), sticks or in the form of the aforementioned liquid or viscous emulsions.

The composition according to the invention contains at least one effect-imparting constituent. The constituents mentioned may especially have a coloring effect or else provide other effects, such as glitter and/or metallic effects. The composition according to the invention preferably comprises at least one colorant which is preferably selected from the group of lipophilic dyes, hydrophilic dyes, pigments and nacre. Particularly advantageously in accordance with the invention, the concentration of colorants is 0.01% to 40% by weight, particularly advantageously 1.0% to 30% by weight, very particularly advantageously from 2.0% to 25% by weight, based in each case on the total weight of the composition.

For example, it is possible to use lipophilic dyes, such as Sudan I (yellow), Sudan II (orange), Sudan III (red), Sudan IV (scarlet), DC Red 17, DC Green 6, β-carotene, soybean oil, DC Yellow 11, DC Violet 2, DC Orange 5 and DC Yellow 10.

The pigments may in principle be any inorganic or organic pigments which are used in cosmetic or dermatological compositions. The pigments used in accordance with the invention may, for example, be white or colored, and they may be encased or coated with a hydrophobic treatment agent or be uncoated.

Advantageously, the pigments are selected from the group of the metal oxides, such as the oxides of iron (especially the oxides that are yellow, red, brown or black in color), titanium dioxide, zinc oxide, cerium oxide, zirconium oxide, chromium oxide; manganese violet, ultramarine blue, Prussian blue, ultramarine and iron blue, bismuth oxide chloride, nacre, mica pigments coated with titanium or bismuth oxide chloride, colored pearlescent pigments, for example titanium-mica pigments comprising iron oxides, titanium-mica pigments, especially comprising iron blue or chromium oxide, titanium-mica pigments comprising an organic pigment of the aforementioned type, and pearlescent pigments based on bismuth oxide chloride, carbon black, the pigments of the D&C type, and the coating materials based on cochineal red, barium, strontium, calcium and aluminum, and mixtures thereof.

Particularly advantageously used are the pigments of iron oxides or titanium dioxide.

For better wettability of the pigments by the fatty phase oils, the surface of the pigments is preferably treated with a hydrophobic treatment agent. The hydrophobic treatment agent is preferably selected from the group of silicones, such as methicone, dimethicone, perfluoroalkylsilanes; fatty acids such as stearic acid; metal soaps such as aluminum dimyristate, the aluminum salt of hydrogenated tallow glutamate, perfluoroalkyl phosphates, perfluoroalkylsilanes, perfluoroalkylsilazanes, hexafluoropropylene polyoxides, polyorganosiloxanes comprising perfluoroalkyl perfluoropolyether groups, amino acids; N-acylated amino acids or salts thereof; lecithin, isopropyl triisostearyl titanate and mixtures thereof. The N-acylated amino acids may comprise an acyl group having 8 to 22 carbon atoms, for example 2-ethylhexanoyl, caproyl, lauroyl, myristoyl, palmitoyl, stearoyl or cocoyl. The salts of these compounds may be aluminum salts, magnesium salts, calcium salts, zirconium salts, tin salts, sodium salts or potassium salts. The amino acid may be, for example, lysine, glutamic acid or alanine.

The decorative cosmetic compositions of the invention may contain one or more emulsifiers or surface-active agents.

Thus, oil-in-water emulsions (O/W) of the invention preferably contain at least one emulsifier having an HLB value >7 and optionally a coemulsifier.

The following non-ionic emulsifiers are used advantageously:
a) fatty acid partial esters and fatty acid esters of polyhydric alcohols and ethoxylated derivatives thereof (for example glyceryl monostearate, sorbitan stearate, glyceryl stearyl citrate, sucrose stearate)
b) ethoxylated fatty alcohols and fatty acids.

Particularly advantageous non-ionic O/W emulsifiers are ethoxylated fatty alcohols or fatty acids, preferably PEG-100 stearate, PEG-40 stearate, ceteareth-20, ceteth-20, steareth-20, ceteareth-12, ceteth-12, steareth-12 and esters of mono-, oligo- or polysaccharides with fatty acids, preferably cetearyl glucoside, methyl glucose distearate.

Advantageous anionic emulsifiers are soaps, for example sodium or triethanolamine salts of stearic acid or palmitic acid, and esters of citric acid such as glyceryl stearate citrate.

Suitable coemulsifiers used for O/W emulsions according to the invention may be fatty alcohols having 8 to 30 carbon atoms, monoglyceryl esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol, and polyethylene glycol (2) stearyl ether (steareth-2).

It may be advantageous in the context of the present invention to use further emulsifiers. This may be done, for example, to further increase the water resistance of the preparations of the invention.

Examples of suitable emulsifiers are alkyl methicone copolyols and alkyl dimethicone copolyols, in particular cetyl dimethicone copolyol, lauryl methicone copolyol, W/O emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4 isostearate, acrylate/C₁₀₋₃₀ alkyl acrylate crosspolymer, sorbitan isostearate, poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoyl polyglyceryl-3 diisostearate, glycol distearate, and polyglyceryl-3 dipolyhydroxystearate.

The O/W compositions according to the invention may advantageously contain thickeners for the water phase. Advantageous thickeners are:
crosslinked or non-crosslinked acrylic acid or methacrylic acid homo- or copolymers. These include crosslinked homopolymers of methacrylic acid or acrylic acid, copolymers of acrylic acid and/or methacrylic acid and monomers derived from other acrylic or vinyl monomers, such as C10-30 alkyl acrylates, C10-30 alkyl methacrylates and vinyl acetate.
thickening polymers of natural origin, for example based on cellulose, guar gum, xanthan, scleroglucan, gellan gum, rhamsan and karaya gum, alginates, maltodextrin, starch and derivatives thereof, locust bean gum, hyaluronic acid, carrageenan, polysaccharides.
non-ionic, anionic, cationic or amphoteric associative polymers, for example based on polyethylene glycols and derivatives thereof, or polyurethanes.
crosslinked or noncrosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid or methacrylic acid homo- or copolymers and crosslinked copolymers of 2-acrylamido-2-methylpropanesulfonic acid.

Very particularly advantageous thickeners are xanthan gum, such as the products supplied under the Keltrol® and Kelza® names by CP Kelco or the products from RHODIA with the Rhodopol® name and guar gum, such as the products available under the Jaguar® HP105 name by RHODIA.

Very particularly advantageous thickeners are likewise crosslinked homopolymers of methacrylic acid or acrylic acid, which are commercially available from Lubrizol under the Carbopol® 940, Carbopol® 941, Carbopol® 980, Carbopol® 981, Carbopol® ETD 2001, Carbopol® EDT 2050, Carbopol 2984, Carbopol® 5984 and Carbopol® Ultrez 10 names, from 3V under the Synthalen® K, Synthalen® L and Synthalen® MS names, and from PROTEX under the Modarez® V 1250 PX, Modarez® V2000 PX, Viscaron® A1600 PE and Viscaron® A700 PE names.

Very particularly advantageous thickeners are crosslinked copolymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such polymers are commercially available, for example, from Lubrizol under the Carbopol® 1342, Carbopol® 1382, Pemulen® TR1 or Pemulen® TR2 names and from Ashland under the Ultrathix® P-100 (INCI: Acrylic Acid/VP Crosspolymer) names.

Very particularly advantageous thickeners are crosslinked copolymers of 2-acrylamido-2-methylpropanesulfonic acid. Such copolymers are available, for example, from Clariant under the Aristoflex® AVC names (INCI: Ammonium Acryloyldimethyltaurate/VP Copolymer).

These thickeners are generally present at a concentration of about 0% to 2% by weight, preferably 0% to 1% by weight, based on the total weight of the composition according to the invention.

Further compositions according to the invention may be water-in-oil or water-in-silicone emulsions. Preference is given to water-in-oil (W/O) or water-in-silicone emulsions (W/Si) comprising one or more silicone emulsifiers (W/S) having an HLB value ≤8 or one or more W/O-emulsifiers having an HLB value <7 and optionally one or more O/W emulsifiers having an HLB value >10.

The silicone emulsifiers may advantageously be selected from the group comprising alkyl dimethicone copolyols, such as cetyl PEG/PPG 10/1 dimethicone copolyol (ABIL® EM 90 from Degussa) or lauryl PEG/PPG-18/18 dimethicone (Dow Corning® 5200 from Dow Corning Ltd.), and dimethicone copolyols such as PEG-10 dimethicone (KF-6017 from Shin Etsu), PEG/PPG-18/18 dimethicone (Dow Corning 5225C from Dow Corning Ltd.) or PEG/PPG-19/19 dimethicone (Dow Corning BY-11 030 from Dow Corning Ltd.).

W/O emulsifiers having an HLB value <7 may advantageously be selected from the group comprising sorbitan stearate, sorbitan oleate, glyceryl isostearate, polyglyceryl-3 oleate, pentaerythrityl isostearate, methylglucose dioleate, PEG-7 hydrogenated castor oil, polyglyceryl-4 isostearate, hexyl laurate, sorbitan isostearate, polyglyceryl-2 dipolyhydroxystearate, polyglyceryl-3 diisostearate, PEG-30 dipolyhydroxystearate, diisostearoyl polyglyceryl-3 diisostearate, polyglyceryl-3 dipolyhydroxystearate, polyglyceryl-4 dipolyhydroxystearate, polyglyceryl-3 dioleate, and wool wax alcohol (Eucerit).

O/W emulsifiers having an HLB value >10 may advantageously be selected from the group comprising lecithin, trilaureth-4 phosphate, polysorbate 20, polysorbate 60, PEG-22-dodecyl glycol copolymer, sucrose stearate, and sucrose laurate.

For stabilization of the W/O emulsion according to the invention against sedimentation or flocculation of water droplets, an oil thickener may advantageously be used.

Particularly advantageous oil thickeners are organomodified clays such as organomodified bentonites (Bentone® 34 from Rheox), organomodified hectorites (Bentone® 27 and Bentone® 38 from Rheox) or organomodified montmorillonite, hydrophobic fumed silica, in which the silanol groups are substituted by trimethylsiloxy groups (Aerosil® R812 from Degussa) or with dimethylsiloxy groups or polydimethylsiloxane (Aerosil® R972, Aerosil® R974 from Degussa, Cab-O-Sil® TS-610, Cab-O-Sil® TS-720 from Cabot), magnesium or aluminum stearate, or styrene copolymers such as styrene-butadiene-styrene, styrene-isopropene-styrene, styrene-ethylene/butene-styrene or styrene-ethylene/propene-styrene.

The thickener for the fatty phase may be present in an amount of 0.1% to 5% by weight, based on the total weight of the emulsion, and better yet 0.4% to 3% by weight.

The aqueous phase may additionally comprise stabilizing agents. The stabilizing agent may be, for example, sodium chloride, magnesium chloride or magnesium sulfate, and mixtures thereof.

Oils may be used in W/O, W/Si and O/W emulsions.

If present, the fatty phase of the composition of the invention comprises at least one non-volatile oil. The fatty phase of the composition may also further comprise volatile oils and waxes. The O/W composition advantageously comprises 0% to 45% by weight of oils, based on the total weight of the composition, and particularly advantageously 0% to 20% by weight of oils. The W/O or W/Si composition advantageously comprises at least 20% by weight of oils, based on the total weight of the composition.

The non-volatile oil is advantageously selected from the group consisting of mineral, animal, plant or synthetic origin, polar or non-polar oils, and mixtures thereof.

Polar oils may be selected from among the lecithins and the fatty acid triglycerides, namely the triglyceryl esters of saturated and/or unsaturated, branched and/or unbranched alkanecarboxylic acids having a chain length of 8 to 24, especially 12 to 18, carbon atoms. For example, the fatty acid triglycerides may be selected from the group consisting of cocoglyceride, olive oil, sunflower oil, soybean oil, peanut oil, rapeseed oil, almond oil, palm oil, coconut oil, castor oil, wheatgerm oil, grapeseed oil, safflower oil, evening primrose oil, macadamia nut oil, apricot kernel oil, avocado oil, and the like.

Further advantageous polar oils may be selected from the group of esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, and also from the group of esters of aromatic carboxylic acids and saturated and/or unsaturated, branched and/or unbranched alcohols having a chain length of 3 to 30 carbon atoms. For example, the ester oils may preferably be selected from the group consisting of phenethyl benzoate, octyl palmitate, octyl cocoate, octyl isostearate, octyldodecyl myristate, octyl dodecanol, cetearyl isononanoate, isopropyl myristate, isopropyl palmitate, isopropyl stearate, isopropyl oleate, n-butyl stearate, n-hexyl laurate, n-decyl oleate, diisopropyl adipate, isooctyl stearate, isononyl stearate, isononyl isononanoate, 2-ethylhexyl palmitate, 2-ethylhexyl laurate, 2-hexyldecyl laurate, 2-hexyldecyl stearate, 2-octyldodecyl palmitate, 2-octyldodecyl myristate, 2-octyldodecyl lactate, 2-diethylhexyl succinate, diisostearyl malate, glyceryl triisostearate, diglyceryl triisostearate, stearyl heptanoate, oleyl oleate, oleyl erucate, erucyl oleate, erucyl erucate, tridecyl stearate, tridecyl trimellitate, and also synthetic, semisynthetic, and natural mixtures of such esters, for example jojoba oil.

The polar oils may advantageously be selected from the group of the dialkyl ethers and dialkyl carbonates; advantageous examples are dicaprylyl ether (Cetiol® OE from BASF Personal Care & Nutrition GmbH) and/or dicaprylyl carbonate (for example Cetiol® CC from BASF Personal Care & Nutrition GmbH).

It is further preferable for the polar oils to be selected from the group consisting of isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, C12-15-alkyl benzoate, myristyl myristate, isodecyl neopentanoate, triisostearin, dipentaerythrityl hexacaprylate/hexacaprate, propylene glycol monoisostearate, tricaprylin, dimethyl isosorbide, butyloctyl salicylate (as obtainable for example under the trade name Hallbrite® BHB from CP Hall), hexadecyl benzoate and butyloctyl benzoate and mixtures thereof (Hallstar® AB) and/or diethylhexyl naphthalate (Hallbrite® TQ or Corapan® TQ from Symrise).

The non-volatile oil may likewise advantageously also be a non-polar oil selected from the group of the branched and unbranched hydrocarbons, in particular mineral oil, vaseline oil, paraffin oil, squalane and squalene, polyolefins, for example polydecenes, hydrogenated polyisobutenes, C13-16 isoparaffin and isohexadecane.

The non-polar non-volatile oil may be selected from the non-volatile silicone oils.

The non-volatile silicone oils may include the polydimethylsiloxanes (PDMS) that are optionally phenylated, such as phenyltrimethicone, or are optionally substituted by aliphatic and/or aromatic groups or by functional groups, for example hydroxyl groups, thiol groups and/or amino groups; polysiloxanes modified with fatty acids, fatty alcohols or polyoxyalkylenes, and mixtures thereof.

The composition according to the invention may further comprise a wax.

In the context of the present document, a wax is defined as a lipophilic fatty substance that is solid at room temperature (25° C.) and shows a reversible solid/liquid change of state at a melting temperature between 30° C. and 200° C. Above the melting point, the viscosity of the wax becomes low and it becomes miscible with oils.

The wax is advantageously selected from the groups of natural waxes, for example cotton wax, carnauba wax, candelilla wax, esparto wax, Japan wax, montan wax, sugarcane wax, beeswax, wool wax, shellac, microwaxes, ceresin, ozokerite, ouricury wax, cork fiber wax, lignite waxes, berry wax, shea butter, or synthetic waxes such as paraffin waxes, polyethylene waxes, waxes produced by Fischer-Tropsch synthesis, hydrogenated oils, fatty acid esters and glycerides that are solid at 25° C., silicone waxes and derivatives (alkyl derivatives, alkoxy derivatives and/or esters of polymethylsiloxane) and mixtures thereof. The waxes can be in the form of stable dispersions of colloidal wax particles which can be produced by known processes, for example according to “Microemulsions Theory and Practice”, L. M. Prince Ed., Academic Press (1977), pages 21-32.

The waxes may be present in amounts of 0% to 30% by weight, based on the total weight of the composition, and preferably 0% to 20% by weight.

The composition according to the invention may further comprise a volatile oil selected from the group consisting of volatile hydrocarbons, siliconized oils, and fluorinated oils.

The volatile oil may be present in an amount of 0% to 25% by weight, based on the total weight of the emulsion, preferably 0% to 20% by weight, and more preferably 0% to 15% by weight.

In the context of the present document, a volatile oil is an oil which evaporates within less than one hour on contact with the skin at room temperature and atmospheric pressure. The volatile oil is liquid at room temperature and, at room temperature and atmospheric pressure, has a vapor pressure of 0.13 to 40 000 Pa (10⁻³ to 300 mm Hg), preferably 1.3 to 13 000 Pa (0.01 to 100 mm Hg) and more preferably 1.3 to 1300 Pa (0.01 to 10 mm Hg), and a boiling point of 150 to 260° C. and preferably 170 to 250° C.

A hydrocarbon oil is understood as meaning an oil that is formed essentially from carbon atoms and hydrogen atoms, and optionally oxygen atoms or nitrogen atoms, and does not contain any silicon atoms or fluorine atoms; it may also consist of carbon atoms and hydrogen atoms, but it may also contain ester groups, ether groups, amino groups or amide groups.

A silicone oil is understood as meaning an oil containing at least one silicon atom and especially Si—O groups, such as polydiorganosiloxanes in particular.

A fluorinated oil is understood as meaning an oil containing at least one fluorine atom. The volatile hydrocarbon oil of the invention may be selected from among the hydrocarbon oils having a flash point of 40 to 102° C., preferably 40 to 55° C., and more preferably 40 to 50° C.

Examples of the volatile hydrocarbon oils are those having 8 to 16 carbon atoms and mixtures thereof, in particular branched C₈₋₁₆ alkanes such as isoalkanes (also referred to as isoparaffins) having 8 to 16 carbon atoms, isododecane, isodecane, isohexadecane and, for example, the oils marketed under the Isopars® or Permetyls® trade names; and the branched C₈₋₁₆ esters, such as isohexyl neopentanoate, and mixtures thereof.

Particularly advantageous are volatile hydrocarbon oils such as isododecane, isodecane, and isohexadecane.

The volatile siliconized oil according to the invention may be selected from among the siliconized oils having a flash point of 40 to 102° C., preferably a flash point exceeding 55° C. and not more than 95° C. and particularly preferably in the range from 65 to 95° C.

For example, the volatile siliconized oils are straight-chain or cyclic silicone oils having 2 to 7 silicon atoms, these silicones optionally containing alkyl or alkoxy groups having 1 to 10 carbon atoms.

Particularly advantageous are volatile siliconized oils such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

The volatile fluorinated oil does not generally have a flash point.

For example, the volatile fluorinated oils are nonafluoroethoxybutane, nonafluoromethoxybutane, decafluoropentane, tetradecafluorohexane, dodecafluoropentane and mixtures thereof.

The preferred cosmetically acceptable medium of the composition of the invention comprises water and optionally a cosmetically acceptable water-miscible suitable organic solvent.

The water used in the composition according to the invention may be a blossom water, pure demineralized water, mineral water, thermal water, and/or seawater.

In the case of an O/W composition as the composition according to the invention, the water content may be in the range from 40% to 95% by weight, preferably in the range from 50% to 90% by weight, most preferably in the range from 60% to 80% by weight, based on the total weight of the composition. In the case of a W/O composition, the water content is in the range from 0% to 60% by weight, preferably in the range from 10% to 50% by weight, most preferably in the range from 30% to 50% by weight, based on the total weight of the composition.

The preferred solvents are, for example, the aliphatic alcohols having C1-4 carbon atoms, such as ethanol and isopropanol; polyol and derivatives thereof such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, and mixtures thereof.

The proportion of the solvent or solvents in the composition of the invention may, for example, be in the range from 0% to 25% by weight and preferably 0% to 15% by weight, based on the total weight of the composition.

Further compositions of the invention maybe a loose powder or a compact powder.

The decorative cosmetic composition according to the invention may preferably also provide what is known as a foundation effect, by means of which skin unevennesses, such as wrinkles etc., can be smoothed.

The decorative cosmetic compositions according to the invention may contain further additives that are customary in cosmetics, such as for example: antioxidants, light stabilizers and/or other auxiliaries and additives such as emulsifiers, surface-active substances, defoamers, thickeners, surfactants, active ingredients, moisturizers, sensory additives, UV filters, film formers, solvents, coalescing agents, flavorings, odor absorbers, perfumes, gel formers and/or other polymer dispersions such as dispersions based on polyacrylates, fillers, plasticizers, pigments, leveling agents and/or thixotropic agents, emollients, preservatives. The amounts of the various additives are known to those skilled in the art for the range to be used, and are for example in the range from 0% to 25% by weight, based on the total weight of the composition.

The decorative cosmetic composition according to the invention may also comprise sensory additives. Sensory additives are understood to mean colorless or white, mineral or synthetic, lamellar, spherical or elongate inert particles or a non-particulate sensory additive which, for example, further improve the sensory properties of the formulations and, for example, leave the skin feeling velvety or silky.

The sensory additives may be present in the composition according to the invention, for example, in an amount of 0% to 10% by weight, based on the total weight of the composition, and preferably of 0% to 7%.

Advantageous particulate sensory additives in the context of the present invention are talc, mica, silicon dioxide, kaolin, starch and derivatives thereof (for example tapioca starch, di-starch phosphate, aluminum starch or sodium starch octenylsuccinate and the like), fumed silica, pigments having neither principally UV filter action nor coloring action (for example boron nitride, etc.), boron nitride, calcium carbonate, dicalcium phosphate, magnesium carbonate, magnesium hydrogencarbonate, hydroxyapatites, microcrystalline cellulose, powders of synthetic polymers such as polyamides (for example the polymers available under the “Nylon®” trade name), polyethylene, poly-β-alanine, polytetrafluoroethylene (“Teflon®”), polyacrylate, polyurethane, lauroyllysines, silicone resin (for example the polymers available under the “Tospearl®” trade name from Kobo Products Inc.), hollow particles of polyvinylidene/acrylonitriles (Expancel® from Akzo Nobel) or hollow particles of silicon dioxide (Silica Beads® from MAPRECOS).

Advantageous non-particulate sensory additives may be selected from the group of dimethiconols (for example Dow Corning 1503 Fluid from Dow Corning Ltd.), silicone copolymers (for example divinyldimethicone/dimethicone copolymer, Dow Corning HMW 2220 from Dow Corning Ltd.), or silicone elastomers (for example dimethicone crosspolymer, Dow Corning 9040 Silicone Elastomer Blend from Dow Corning Ltd).

The composition according to the invention may optionally also comprise sunscreen filters, the total amount of sunscreen filters being from 0% by weight to 30% by weight, advantageously from 0% by weight to 20% by weight, particularly advantageously from 0% by weight to 10% by weight, based on the total weight of the composition according to the invention. The sunscreen filters (or UV filters) may in particular be selected from among the organic filters, the physical filters and mixtures thereof.

The composition according to the invention may comprise UV-A filters, UV-B filters or broad-spectrum filters. The UV filters used may be oil-soluble or water-soluble. The appended list of UV filters mentioned below is of course not limiting.

Examples of UV-B filters include:
(1) salicylic acid derivatives, particularly homomenthyl salicylate, octyl salicylate and 4-isopropylbenzyl salicylate;
(2) cinnamic acid derivatives, especially 2-ethylhexyl p-methoxycinnamate, available from DSM under the Parsol MCX® name, and isopentyl 4-methoxycinnamate;
(3) liquid β,β′-diphenylacrylate derivatives, especially 2-ethylhexyl α,β′-diphenylacrylate or octocrylene, available from BASF under the UVINUL N539® name;
(4) p-aminobenzoic acid derivatives, especially 2-ethylhexyl 4-(dimethylamino)benzoate, amyl 4-(dimethylamino)benzoate;
(5) 3-benzylidenecamphor derivatives, especially 3-(4-methylbenzylidene)camphor, commercially available from Merck under the EUSOLEX 6300® name, 3-benzylidenecamphor, benzylidenecamphorsulfonic acid and polyacrylamidomethylbenzylidenecamphor;
(6) 2-phenylbenzimidazole-5-sulfonic acid available under the EUSOLEX 232® name from Merck;
(7) 1,3,5-triazine derivatives, in particular: —2,4,6-tris[p-(2′-ethylhexyl-1′-oxycarbonyl)anilino]-1,3,5-triazine, supplied by BASF under the UVINUL T150® name, and -dioctylbutamidotriazone, supplied by Sigma 3V under the UVASORB HEB® name;
(8) esters of benzalmalonic acid, especially di(2-ethylhexyl) 4-methoxybenzalmalonate and 3-(4-(2,2-bisethoxycarbonylvinyl)phenoxy)propenyl)methoxysiloxane/dimethylsiloxane copolymer, available from DSM under the Parsol® SLX name; and
(9) mixtures of these filters.

Examples of UV-A filters include:
(1) dibenzoylmethane derivatives, particularly 4-(t-butyl)-4′-methoxydibenzoylmethane, which is supplied by DSM under the PARSOL 1789® name, and 1-phenyl-3-(4′-isopropylphenyl)propane-1,3-dione;
(2) benzene-1,4-[di(3-methylidenecamphor-10-sulfonic acid)], optionally fully or partly neutralized, commercially available under the MEXORYL SX® name from Chimex.
(3) hexyl 2-(4′-diethylamino-2′-hydroxybenzoyl)benzoate (also aminobenzophenone);
(4) silane derivatives or polyorganosiloxanes having benzophenone groups;
(5) anthranilates, particularly menthyl anthranilate, which is supplied by Symrise under the NEO HELIOPAN MA® name;
(6) compounds containing at least two benzazolyl groups or at least one benzodiazolyl group per molecule, especially 1,4-bis(benzimidazolyl)phenylene-3,3′,5,5′-tetrasulfonic acid and salts thereof, commercially available from Symrise;
(7) silicon derivatives of benzimidazolylbenzazoles that are N-substituted, or of benzofuranylbenzazoles, especially: -2-[1-[3-[1,3,3,3-tetramethyl-1-[(trimethylsilyl)oxy]/disiloxanyl]propyl]-1H-benzimidazol-2-yl]benzoxazole; -2-[1-[3-[1,3,3,3-tetramethyl-1-[(trimethylsilyl)oxy]disiloxanyl]propyl]-1H-benzimidazol-2-yl]benzothiazole; -2-[1-(3-trimethylsilanylpropyl)-1H-benzimidazol-2-yl]benzoxazole; -6-methoxy-1,1′-bis(3-trimethylsilanylpropyl)-1H,1′H-[2,2′]dibenzimidazolylbenzoxazole; -2-[1-(3-trimethylsilanylpropyl)-1H-benzimidazol-2-yl]benzothiazole; which are described in patent application EP-A-1 028 120;
(8) triazine derivatives, especially 2,4-bis[5-1(dimethylpropyl)benzoxazol-2-yl-(4-phenyl)iminol-6-(2-ethylhexyl)imino-1,3,5-triazine, supplied by 3V under the Uvasorb®K2A name; and
(9) mixtures thereof.

Examples of broad-spectrum filters are:
(1) benzophenone derivatives, for example
   2,4-dihydroxybenzophenone (Benzophenone-1);
   2,2′,4,4′-tetrahydroxybenzophenone (Benzophenone-2);
   2-hydroxy-4-methoxybenzophenone (Benzophenone-3), available from BASF under the UVINUL M40® name;
   2-hydroxy-4-methoxybenzophenone-5-sulfonic acid (Benzophenone-4), and the sulfonate form thereof (Benzophenone-5), commercially available from BASF under the UVINUL MS40® name;
   2,2′-dihydroxy-4,4′-dimethoxybenzophenone (Benzophenone-6);
   5-chloro-2-hydroxybenzophenone (Benzophenone-7);
   2,2′-dihydroxy-4-methoxybenzophenone (Benzophenone-8);
   the disodium salt of 2,2′-dihydroxy-4,4′-dimethoxybenzophenone-5,5′-disulfonic acid (Benzophenone-9);
   2-hydroxy-4-methoxy-4′-methylbenzophenone (Benzophenone-10);
   Benzophenone-11;
   2-hydroxy-4-(octyloxy)benzophenone (Benzophenone-12).
(2) triazine derivatives, especially 2,4-bis{[4-2-ethylhexyloxy)-2-hydroxy]phenyl}-6-(4-methoxyphenyl)-1,3,5-triazine, which is supplied by BASF under the TINOSORB S® name, and 2,2′-methylenebis[6-(2H-benzotriazol-2-yl)-4-(1,1,3,3-tetramethylbutyl)phenol], available from BASF under the TINOSORB M® name; and
(3) 2-(1H-benzotriazol-2-yl)-4-methyl-6-[2-methyl-3-[1,3,3,3-tetramethyl-1-[(trimethylsilyl)oxy]disiloxanyl]propyl]phenol with the INCI name Drometrizole Trisiloxane.

It is also possible to use a mixture of two or more filters and a mixture of UV-B filters, UV-A filters and broad-spectrum filters, and also mixtures with physical filters.

The physical filters may include the sulfate of barium, and oxides of titanium (titanium dioxide, amorphous or crystalline in the form of rutile and/or anatase), of zinc, of iron, of zirconium, of cerium, silicon, manganese or mixtures thereof. The metal oxides may be in particle form with a size in the micrometer range or nanometer range (nanopigments). The mean particle sizes for the nanopigments are, for example, 5 to 100 nm.

The decorative cosmetic composition of the invention may comprise one or more humectants (moisturizers).

Particularly advantageous moisturizers in the context of the present invention are, for example, glycerol, polyglycerol, sorbitol, dimethyl isosorbide, lactic acid and/or lactates, especially sodium lactate, butylene glycol, propylene glycol, biosaccharide gum 1, glycine soya, hydroxyethylurea, ethylhexyloxyglycerol, pyrrolidonecarboxylic acid and urea. In addition, it is especially advantageous to use polymeric moisturizers from the group of the water-soluble and/or water-swellable polysaccharides and/or those that can be gelated with the aid of water. Especially advantageous are, for example, hyaluronic acid, chitosan and/or a fucose-rich polysaccharide available under the Fucogel™ 1000 name from SOLABIA S.A.

In the context of the present invention, it is particularly advantageously possible to use water-soluble antioxidants, such as vitamins, e.g. ascorbic acid and derivatives thereof. Vitamin E and derivatives thereof and vitamin A and derivatives thereof are especially advantageous.

Other advantageous active ingredients in the composition according to the invention are α-hydroxy acids such as glycolic acid, lactic acid, malic acid, tartaric acid, citric acid and mandelic acid, β-hydroxy acids such as salicylic acid and acylated derivatives thereof, 2-hydroxyalkanoic acids and derivatives thereof; natural active ingredients and/or derivatives thereof, such as alpha-lipoic acid, folic acid, phytoene, D-biotin, coenzyme Q10, alpha-glucosylrutin, carnitine, carnosine, natural and/or synthetic isoflavonoids, creatine, creatinine, taurine and/or [beta]-alanine and 8-hexadecene-1,16-dicarboxylic acid (dioic acid, CAS number 20701-68-2; provisional INCI name Octadecenedioic Acid) and/or licochalcone A and plant extracts.

Advantageous film formers are trimethylsiloxysilicates, silicone acrylate copolymers (e.g. TIB4-200 from Dow Corning or KP-561 from Shin Etsu), trimethyl pentaphenyl trisiloxanes (Dow Corning 555 Cosmetic Fluid from Dow Corning Ltd.) or vinylpyrrolidone copolymer (e.g. PVP/eicosene copolymer or PVP/hexadecane copolymer).

The present invention is elucidated using examples, which are not to be understood as being limiting. All amounts, proportions and percentages, unless stated otherwise, are based on weight and the total amount or total weight of the compositions.

### EXAMPLES

Materials Used:

PUD 1 (comparative): Polyurethane dispersion (solids content 40% by weight) based on synthetically produced polyester polyol 1, dicyclohexylmethane diisocyanate, ethylenediamine and aminoethanesulfonic acid sodium salt, polyurethane is 100 mol % synthetic.

PUD 2 (according to the invention): Polyurethane dispersion (solids content 30% by weight) based on bio-based polyester polyol 2, isophorone diisocyanate, isophoronediamine and aminoethanesulfonic acid sodium salt, polyurethane is 55 mol % bio-based. The polyurethane present in PUD 2 corresponds to Polyurethane-93 according to INCI nomenclature.

PUD 3 (according to the invention): Polyurethane dispersion (solids content 40% by weight) based on bio-based polyester polyol 2, dicyclohexylmethane diisocyanate, ethylenediamine and aminoethanesulfonic acid sodium salt, polyurethane is 60 mol % bio-based. The polyurethane present in PUD 3 corresponds to Polyurethane-99 according to INCI nomenclature.

Polyester polyol 1 (comparative): synthetically produced from adipic acid, neopentyl glycol and hexane-1,6-diol

Polyester polyol 2: (according to the invention): bio-based polyester polyol formed from succinic acid, butane-1,4-diol and neopentyl glycol

DSC Measurements:

The glass transition temperatures Tg of the comparative polyester polyol 1 and of the polyester polyol 2 according to the invention, and also of the comparative polyurethane dispersions PUD 1 and of the polyurethane dispersions PUD 2 and PUD 3 according to the invention, in each case as a dried film, were measured by the following method:

Physical transformations such as melting points or glass transition temperatures T_{g} are determined by measuring the heat capacity as a function of temperature using a DSC3+e calorimeter from Mettler-Toledo. The temperature and the enthalpy of fusion are calibrated using n-heptane, indium, lead and zinc. The purge gas used is nitrogen at a flow rate of 20 ml/min. Cooling is effected by means of liquid nitrogen. The temperature gradient is 20 K/min. Measurements are taken in the temperature range between −70 and +150° C. (2 heating runs).

The measurements are taken on the form as supplied, i.e. directly on the aqueous polyurethane dispersions, without preconditioning. The sample weights are between 10 and 12 mg of sample mass in a hermetically sealable perforated aluminum standard crucible.

Evaluation: the glass transition temperature T_{g} was determined as the temperature at half height of the glass transition on the second heating run.

The results of the measurements are shown in table 1.

TABLE 1
DSC results

| No. | Name | Tg [° C.] |
|---|---|---|
| C1 | Polyester polyol 1 | −60.1 |
| 2 | Polyester polyol 2 | −38.6 |
| C3 | PUD 1 | −44.1 |
| 4 | PUD 2 | −16.5 |
| 5 | PUD 3 | −21.0 |

C Comparative

Use in Mascara:

The composition of the test mascara is given in table 2:

TABLE 2
Test mascara formulation

| | | Amount |
|---|---|---|
| Phase | Component (INCI name) | [% by weight] |
| A | Water | to 100 |
| | Hydroxyethylcellulose | 0.70 |
| | Triethanolamine (99%) | 2.30 |
| | Nylon-66 | 1.50 |
| B | Iron Oxide Black | 8.00 |
| C | Glyceryl stearate | 2.30 |
| | Stearic acid | 5.60 |
| | Beeswax | 7.00 |
| | Carnauba wax | 7.00 |
| | Dimethicone | 0.50 |
| D | Phenoxyethanol | 0.80 |
| | Polyurethane (as solid) | 4.00 |
| Sum total | | 100.00 |

The test mascara was prepared according to the following method:
1. Dissolve ingredients of phase A in water and heat to 80° C.
2. Once phase A was homogeneous, the pigment was added with stirring, the whole mixture was homogenized.
3. In a separate vessel, the ingredients of phase C were mixed and heated to 80° C.
4. Once phase C was homogeneous, phase C was slowly added to phase A/B. The emulsion was homogenized.
5. Phase D was added during cooling.
6. After checking the pH, this was adjusted to pH 6.5 to 7.0 where necessary.

The water resistance of the mascara was tested by the following method:

Two cotton pads were moistened with water heated to 37° C. The mascara formulation was applied 10 times in a zigzag pattern to false eyelashes made from human hair. After drying, the eyelashes were placed between the two cotton pads and pulled from the root to the tip with gentle pressure. The residues on the cotton pads were assessed using the scale in table 3.

TABLE 3
Assessment of water resistance

| Degree of water resistance | Value |
|---|---|
| No residue on cotton pads | 5 |
| Residue area on cotton pads 0 to 1 cm² | 4 |
| Residue area on cotton pads 1 to 2 cm² | 3 |
| Residue area on cotton pads 2 to 4 cm² | 2 |
| Residue area on cotton pads more than 4 cm² | 1 |

The results of the water resistance test are shown in table 4. The higher the value, the better the water resistance.

TABLE 4
Results of water resistance test

| Test | PUD 1 | PUD 2 | PUD 3 |
|---|---|---|---|
| Value | 4 | 5 | 5 |

Use in an Eyeliner:

The eyeliner formulation shown in table 5 was prepared according to the method presented below.

All constituents of phase A were added to a vessel at room temperature with stirring. Once phase A was homogeneous, phase B was added. This mixture was homogenized. Phase C was then added with stirring.

TABLE 5
Eyeliner formulation:

| | | Amount |
|---|---|---|
| Phase | Component (INCI name) | [% by weight] |
| A | Water | to 100 |
| | Xanthan | 0.50 |
| | Microcrystalline (and) Algin | 0.30 |
| B | Iron Oxide (Black) | 10.00 |
| | Sorbitan Laurate (and) Polyglyceryl-4 | 1.50 |
| | Laurate (and) Dilauryl Citrate | |
| C | Propylene glycol | 2.00 |
| | Polyurethane (solid) | 5.00 |
| | Methylpropanediol (and) Caprylyl Glycol | 1.50 |
| | (and) Phenylpropanol | |
| Sum total | | 100.00 |

Method for Assessing Abrasion Resistance:

The corresponding eyeliner formulation was applied as a line to the skin. Once the formulation was dry, the eyeliner line was rubbed 10 times with a foldback clip. The quality of the eyeliner film is assessed as shown in table 6. The higher the value, the better the abrasion resistance.

TABLE 6
Assessment of abrasion resistance

| Assessment of film quality | Value |
|---|---|
| no change in the film | 5 |
| slight scratches on the film | 4 |
| scratches on the film | 3 |
| film partially rubbed off | 2 |
| film (almost completely) rubbed off | 1 |

The results of the abrasion resistance test are shown in table 7:

TABLE 7
Results of abrasion resistance test

| Polyurethane | PUD 1 | PUD 2 |
|---|---|---|
| Value | 3 | 5 |

Use in a Foundation:

A test foundation was prepared with the components shown in table 8 according to the following method.
1. Phase A was predispersed at RT, the thickeners being dispersed individually;
2. The TiO₂ from phase B was added to phase A, then stirred;
3. Phase C was heated to 75° C., the remaining constituents of phase B, i.e. pigment and mica, were added to phase C, and the mixture was homogenized for 10 minutes;
4. The mixture of phases A and B was heated to 75° C., then the mixture of phases C and B was added to the mixture of phases A and B at 75° C., the resulting mixture was stirred and homogenized for 10 min;
5. Phase E was added at 45° C.

TABLE 8
Foundation formulation

| | | Amount |
|---|---|---|
| Phase | Component (INCI name) | [% by weight] |
| A | Water | to 100.00 |
| | Sodium Carboxymethyl cellulose | 0.30 |
| | Magnesium Aluminum Silicate | 0.35 |
| | Polysorbate 20 | 0.40 |
| | Triethanolamine (10%) | 1.25 |
| | Butylene Glycol | 6.00 |
| B | Titanium Dioxide | 4.00 |
| | Red Iron Oxide | 0.27 |
| | Yellow Iron Oxide | 0.54 |
| | Black Iron Oxide | 0.09 |
| | Mica | 1.00 |
| C | Isoeicosane | 9.00 |
| | Isostearic Acid | 1.00 |
| | Stearic Acid | 0.50 |
| | Glyceryl Stearate | 0.50 |
| | Cetyl Stearyl Alcohol | 3.50 |
| | Glyceryl Stearate SE | 2.00 |
| D | Cyclosiloxane | 2.00 |
| | Polymethylsilsesquioxane | 1.00 |
| E | Polyurethane (solid) | 2.00 |
| | Phenoxyethanol (and) Ethylhexylglycerin | 0.80 |
| Sum total | | 100.00 |

0.02 g of the foundation formulation was applied to a defined area of skin using a pipette. The formulation was distributed homogeneously on the skin in a circle (30 circles). 2 subjects evaluated the formulation properties. The evaluation grades were between 0 and 5 for the properties: distribution, stickiness, powdery skin feel, color distribution, roll formation; 5 meaning that the property was fully present.

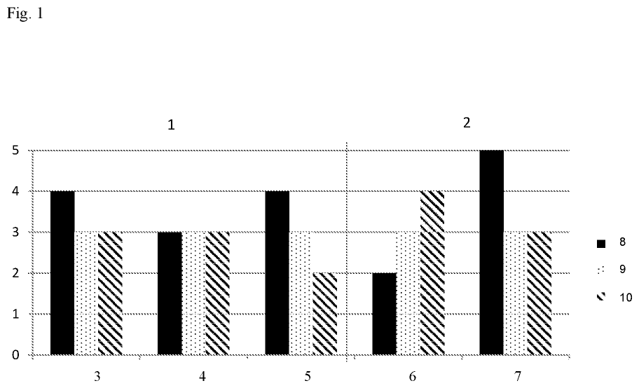

The results of the sensory evaluation are shown in FIG. 1. The references in the figure have the following meanings:
1 before drying
2 after drying
3 distribution
4 stickiness
5 powdery skin feel
6 color distribution
7 roll formation
8 without polyurethane
9 with PUD 1
10 with PUD 2

1. A decorative cosmetic composition, comprising at least one polyurethane obtained by reacting one or more water-insoluble, non-water-dispersible, isocyanate-functional polyurethane prepolymers A with one or more amino-functional compounds B), wherein the polyurethane prepolymer A) is obtained by reacting one or more polyester polyols having a glass transition temperature T_{G} of at least −50° C. and one or more polyisocyanates.

2. The decorative cosmetic composition as claimed in claim 1, wherein one or more isocyanate-functional polyurethane prepolymers A) essentially have neither ionic nor ionogenic groups.

3. The decorative cosmetic composition as claimed in claim 1, wherein the amino-functional compounds B) are selected from primary amines, secondary amines, diamines, and combinations thereof.

4. The decorative cosmetic composition as claimed in claim 1, wherein the amino-functional compounds B) comprise at least one diamine.

5. The decorative cosmetic composition as claimed in claim 1, wherein the amino-functional compounds B) are selected from amino-functional compounds B2) that have ionic and/or ionogenic groups, and amino-functional compounds B1) that do not have any ionic and/or ionogenic groups.

6. The decorative cosmetic composition as claimed in claim 1, wherein the amino-functional compounds B) comprise at least one amino-functional compound B2) that has ionic and/or ionogenic groups.

7. The decorative cosmetic composition as claimed in claim 1, wherein the amino-functional compounds B) comprise at least one amino-functional compound B1) that does not have any ionic and/or ionogenic groups.

8. The decorative cosmetic composition as claimed in claim 1, wherein the amino-functional compounds B) comprise both amino-functional compounds B2) that have ionic and/or ionogenic groups, and amino-functional compounds B1) that do not have any ionic and/or ionogenic groups.

9. The decorative cosmetic composition as claimed in claim 1, wherein the polyurethane comprises at least one sulfonic acid and/or sulfonate group.

10. The decorative cosmetic composition as claimed in claim 1, further comprising one or more constituents that produce a decorative effects.

11. (canceled)

12. A polyurethane obtained by reacting one or more water-insoluble, non-water-dispersible, isocyanate-functional polyurethane prepolymers A with one or more amino-functional compounds B), wherein the polyurethane prepolymer A) is obtained by reacting one or more polyester polyols having a glass transition temperature T_{G} of at least −50° C. and one or more polyisocyanates.

13. A cosmetic method for producing a decorative effect on the skin and/or hair, comprising applying a composition to the skin and/or hair, the composition comprising at least one polyurethane obtained by reacting one or more water-insoluble, non-water-dispersible, isocyanate-functional polyurethane prepolymers A) with one or more amino-functional compounds B), wherein the polyurethane prepolymer A) is obtained by reacting one or more polyester polyols having a glass transition temperature T_{G} of at least −50° C. and one or more polyisocyanates.

14. The cosmetic method as claimed in claim 13, wherein the composition remains on the skin at least to some extent after it has been applied thereto.

15. The decorative cosmetic composition as claimed in claim 6, wherein the at least one amino-functional compound B2) comprises 2-(2-aminoethylamino)ethanesulfonic acid and/or salts thereof.

16. The decorative cosmetic composition as claimed in claim 7, wherein the at least one amino-functional compound B1) comprises a diamine that does not have any ionic and/or ionogenic groups.
